(19) United States
(12) Patent Application Publication
(10) Pub. No.: US 2026/0325418 A1
(43) Pub. Date: Oct. 22, 2026

(54) OIL-IN-WATER COMPOSITION COMPRISING LOW PH COMPONENTS

(71) Applicant: AMOREPACIFIC CORPORATION, Seoul (KR)

(72) Inventors: Shinbeom KANG, Yongin-si (KR); Jeonghwan KIM, Yongin-si (KR); Sungil PARK, Yongin-si (KR); Byungryol PAIK, Yongin-si (KR); Jihyun YOO, Yongin-si (KR); Jongsuk LEE, Yongin-si (KR)

(73) Assignee: AMOREPACIFIC CORPORATION, Seoul (KR)

(21) Appl. No.: 19/566,433

(22) Filed: Mar. 13, 2026

(30) Foreign Application Priority Data
Mar. 26, 2025 (KR) 10-2025-0038789

(51) Int. Cl. A61K 8/34; A61K 8/06; A61K 8/24; A61K 8/37; A61K 8/41; A61K 8/44; A61K 8/49; A61K 8/891; A61Q 19/00

(52) U.S. Cl. CPC A61K 8/345; A61K 8/062; A61K 8/24; A61K 8/342; A61K 8/375; A61K 8/41; A61K 8/416; A61K 8/44; A61K 8/498; A61K 8/891; A61Q 19/00; A61K 2800/30; A61K 2800/33

(57) ABSTRACT

The present invention, in one aspect, discloses an oil-in-water composition including a low pH component. In one aspect, the cosmetic composition of the present invention, even without using a thickener which induces stickiness, includes the active ingredient of a low pH (for example, pH 4 or less), the cationic surfactant, and the fatty acid and/or the fatty alcohol, to form a lamellar gel network structure, whereby it is thickened, and a high viscosity and high strength formulation and a non-sticky and fresh texture can be implemented.

### CROSS REFERENCE TO RELATED APPLICATION

The present application claims priority to Korean Patent Application No. 10-2025-0038789, filed Mar. 26, 2025, the entire contents of which are hereby incorporated by this reference.

### BACKGROUND OF THE INVENTION

### Field of the Invention

The present invention discloses an oil-in-water composition comprising a low pH component.

### Description of the Related Art

The human body internal environment maintains pH 7 to 9 close to neutrality, but the skin generally maintains an acidity of pH 4 to 6, and due to this, between a stratum corneum and an underlying epidermis and dermis, a steep pH gradient of 2 to 3 units is generated.

On the other hand, in O/W (oil-in-water type) sun care formulation and skin care formulation, from a low viscosity (for example, an ampoule type, a lotion type, an essence type) cosmetic to a high viscosity (for example, a cream type) cosmetic, upon application of the low pH component, there is a problem that thickening is not achieved by a thickener (for example, carbomers, acrylics, xanthan gum, natural thickeners, etc.), or pilling occurs, or stickiness which is a negative attribute is induced by use of an excessive amount of the thickener. Accordingly, the stabilized high viscosity cosmetic composition, while including the low pH component in a high content, is limited.

### SUMMARY OF THE INVENTION

An object of the present invention is to provide an oil-in-water composition including a low pH component.

To achieve the object, the present invention, in one aspect, provides an oil-in-water composition including, based on a total oil-in-water composition weight, an active ingredient of pH 4 or less in an amount of 0.1 wt % or more and less than 30 wt %; a surfactant in an amount of exceeding 2.6 wt %; a fatty acid and/or a fatty alcohol in an amount of exceeding 0.3 wt %; a chelating agent in an amount of less than 0.1 wt %; and a polyol, in which the surfactant includes a cationic amine-based surfactant in an amount of exceeding 0.6 wt % and a non-ionic surfactant in an amount of exceeding 0.1 wt %, in which the polyol is at least one polyol selected from the group consisting of sorbitol, maltitol and xylitol, and when the cationic amine-based surfactant is a quaternary amine-based surfactant, the quaternary amine-based surfactant is behentrimonium methosulfate or distearyldimonium chloride.

In one aspect, the cosmetic composition of the present invention, even without using a thickener which induces stickiness, includes the active ingredient of a low pH (for example, pH 4 or less), the cationic surfactant, and the fatty acid and/or the fatty alcohol, to form a lamellar gel network structure, whereby it is thickened, and a high viscosity and high strength formulation and a non-sticky and fresh texture can be implemented. The lamellar structure is a form in which a hydrophobic layer and a hydrophilic layer are alternately and regularly arranged, the hydrophilic layer retains a large amount of water to maintain moisture of the skin, and the hydrophobic layer serves to prevent moisture evaporation. In addition, such enhances a skin protective function along with keratinocytes by regulating mass transfer other than water.

In another aspect, the cosmetic composition of the present invention can implement a stabilized cream formulation despite including the low pH component in a high content, and is excellent in occlusive capability, so that a contact time between the composition and the skin is increased, whereby a skin stratum corneum after application can be maintained at pH 4 to 6 for a long period (for example, 24 hours or more), and thus a stratum corneum of healthy skin can be formed/maintained.

In yet another aspect, the cosmetic composition of the present invention can implement a silky, soft, and smooth finish as if filling skin irregularities, and thus can effectively care for pores and skin texture.

In yet another aspect, the cosmetic composition of the present invention can be utilized as a skin humectant and a skin emollient.

### BRIEF DESCRIPTION OF THE DRAWINGS

FIG. 1 is a polarized light microscope photograph of a cosmetic composition of an embodiment of the present invention. Implementation of a cream formulation is possible by a lamellar gel network structure formation even without a thickener.

FIG. 2 relates to a low pH persistence evaluation.

FIG. 3 relates to a sensory evaluation.

FIG. 4 relates to a moisturizing effect of a low pH active ingredient.

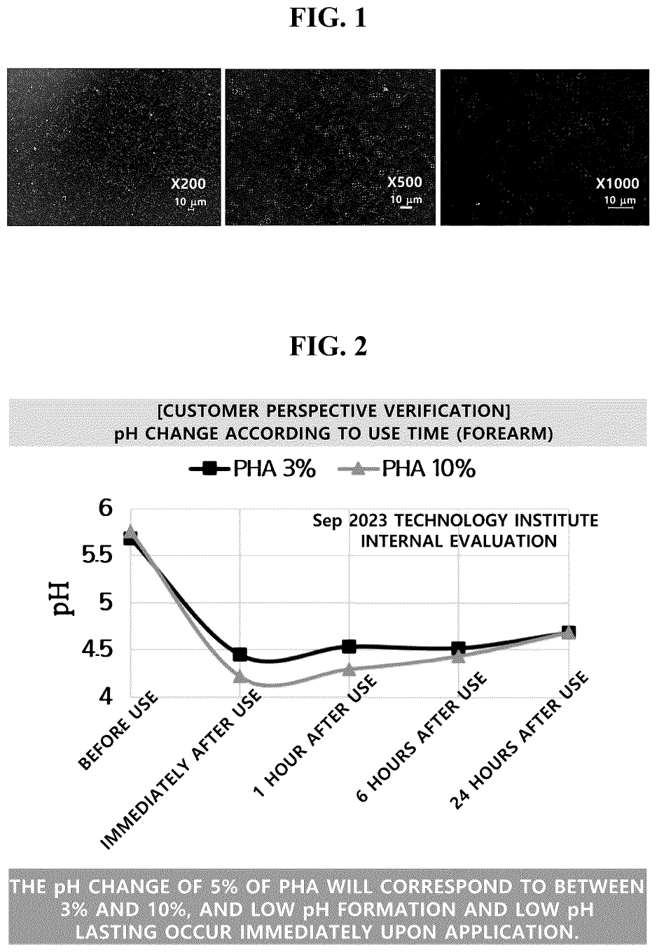

### DETAILED DESCRIPTION OF THE INVENTION

Hereinafter, the present invention will be described in detail.

The present invention, in one perspective, relates to an oil-in-water composition including, based on a total oil-in-water composition weight, an active ingredient of pH 4 or less in an amount of 0.1 wt % or more and less than 30 wt %; a surfactant in an amount of exceeding 2.6 wt %; a fatty acid and/or a fatty alcohol in an amount of exceeding 0.3 wt %; a chelating agent in an amount of less than 0.1 wt %; and a polyol, in which the surfactant includes a cationic amine-based surfactant in an amount of exceeding 0.6 wt % and a non-ionic surfactant in an amount of exceeding 0.1 wt %, in which the polyol is at least one polyol selected from the group consisting of sorbitol, maltitol and xylitol, and when the cationic amine-based surfactant is a quaternary amine-based surfactant, the quaternary amine-based surfactant is behentrimonium methosulfate or distearyldimonium chloride.

In an exemplary embodiment, the composition may include the active ingredient of pH 4 or less in an amount of 0.1 wt % or more and less than 30 wt % based on the total oil-in-water composition weight, and more specifically, the composition may include the active ingredient of pH 4 or less in an amount of 0.1 wt % or more, 0.2 wt % or more, 0.3 wt % or more, 0.4 wt % or more, 0.5 wt % or more, 0.7 wt % or more, 0.8 wt % or more, 0.9 wt % or more, 1.0 wt % or more, 1.5 wt % or more, 2.0 wt % or more, 2.5 wt % or more, 3.0 wt % or more, 3.5 wt % or more, 4.0 wt % or more, 4.5 wt % or more, or 5.0 wt % or more, and less than 30 wt %, less than 29.5 wt %, less than 29 wt %, less than 28.5 wt %, less than 28 wt %, less than 27.5 wt %, less than 27 wt %, less than 26.5 wt %, less than 26 wt %, less than 25.5 wt %, less than 25 wt %, less than 24.5 wt %, less than 24 wt %, less than 23.5 wt %, less than 23 wt %, less than 22.5 wt %, less than 22 wt %, less than 21.5 wt %, less than 21 wt %, or less than 20.5 wt % based on the total oil-in-water composition weight.

In an exemplary embodiment, the active ingredient of pH 4 or less may be at least one selected from the group consisting of poly-hydroxy-acid (PHA) (for example, gluconolactone), beta-hydroxy-acid (BHA) (for example, salicylic acid), alpha-hydroxy-acid (AHA) (for example, glycolic acid, lactic acid, citric acid, malic acid, tartaric acid), pure vitamin C (ascorbic acid), vitamin C derivatives (ethyl ascorbic acid), succinic acid, ferulic acid, aspartic acid, glutamic acid, phenylbenzimidazole sulfonic acid, and terephthalylidene dicamphor sulfonic acid, but is not limited thereto.

In an exemplary embodiment, the surfactant may include a cationic surfactant and/or a non-ionic surfactant.

In an exemplary embodiment, the composition may include the surfactant in an amount of exceeding 2.6 wt % based on the total oil-in-water composition weight, and the surfactant may include a cationic amine-based surfactant in an amount of exceeding 0.6 wt % and a non-ionic surfactant in an amount of exceeding 0.1 wt %. More specifically, the composition may include the cationic surfactant in an amount of exceeding 0.6 wt %, exceeding 0.7 wt %, exceeding 0.8 wt %, exceeding 0.9 wt %, exceeding 1.0 wt %, exceeding 1.1 wt %, exceeding 1.2 wt %, exceeding 1.3 wt %, exceeding 1.4 wt %, or exceeding 1.5 wt %, or 10.0 wt % or less, 9.0 wt % or less, 8.0 wt % or less, 7.0 wt % or less, 6.0 wt % or less, 5.0 wt % or less, 4.9 wt % or less, 4.8 wt % or less, 4.7 wt % or less, 4.6 wt % or less, 4.5 wt % or less, 4.4 wt % or less, 4.3 wt % or less, 4.2 wt % or less, 4.1 wt % or less, or 4.0 wt % or less based on the total oil-in-water composition weight, and may include the non-ionic surfactant in an amount of exceeding 0.1 wt %, exceeding 0.2 wt %, exceeding 0.3 wt %, exceeding 0.4 wt %, exceeding 0.5 wt %, exceeding 0.6 wt %, exceeding 0.7 wt %, exceeding 0.8 wt %, exceeding 0.9 wt %, exceeding 1.0 wt %, exceeding 1.1 wt %, exceeding 1.2 wt %, exceeding 1.3 wt %, exceeding 1.4 wt %, exceeding 1.5 wt %, exceeding 1.6 wt %, exceeding 1.7 wt %, exceeding 1.8 wt %, or exceeding 1.9 wt %, or 20 wt % or less, 19.5 wt % or less, 19 wt % or less, 18.5 wt % or less, 18 wt % or less, 17.5 wt % or less, 17 wt % or less, 16.5 wt % or less, 16 wt % or less, 15.5 wt % or less, 15 wt % or less, 14.5 wt % or less, 14 wt % or less, 13.5 wt % or less, 13 wt % or less, 12.5 wt % or less, 12 wt % or less, 11.5 wt % or less, 11 wt % or less, 10.5 wt % or less, or 10 wt % or less based on the total oil-in-water composition weight.

In an exemplary embodiment, the cationic surfactant may be a tertiary amine-based or a quaternary amine-based surfactant, preferably a tertiary amine-based surfactant. The composition may minimize irritation to the skin by including the tertiary amine-based surfactant. For example, the tertiary amine-based surfactant may be brassicamidopropyl dimethylamine or ricinoleamidopropyl dimethylamine, but is not limited thereto.

In an exemplary embodiment, the non-ionic surfactant may be at least one selected from the group consisting of polyglyceryl-10 stearate (2%), polyglyceryl-10 laurate, polyglyceryl-10 diisostearate, polyglyceryl-10 myristate, polyglyceryl-10 oleate, and polyglyceryl-3 methylglucose distearate, but is not limited thereto.

In an exemplary embodiment, the composition may include the fatty acid and/or the fatty alcohol in an amount of exceeding 0.3 wt % based on the total oil-in-water composition weight, and more specifically, may be exceeding 0.3 wt %, exceeding 0.4 wt %, exceeding 0.5 wt %, exceeding 1.0 wt %, exceeding 1.5 wt %, exceeding 2.0 wt %, exceeding 2.5 wt %, exceeding 3 wt %, exceeding 3.5 wt %, exceeding 4 wt %, exceeding 4.5 wt %, exceeding 5 wt %, exceeding 5.5 wt %, exceeding 6 wt %, exceeding 6.5 wt %, exceeding 7 wt %, exceeding 7.5 wt %, exceeding 8 wt %, or 30 wt % or less, 29 wt % or less, 28 wt % or less, 27 wt % or less, 26 wt % or less, 25 wt % or less, 24 wt % or less, 23 wt % or less, 22 wt % or less, 21 wt % or less, 20 wt % or less, 19 wt % or less, 18 wt % or less, 17 wt % or less, 16 wt % or less, or 15 wt % or less.

In an exemplary embodiment, the fatty acid may be at least one selected from the group consisting of glyceryl stearate (for example, 3%, 2.5%), stearic acid, palmitic acid, myristic acid, lauric acid, caprylic acid, capric acid, arachidic acid, and behenic acid, but is not limited thereto.

In an exemplary embodiment, the fatty alcohol may be at least one selected from the group consisting of cetearyl alcohol (for example, 8%, 6%), cetyl alcohol, arachidyl alcohol, behenyl alcohol, myristyl alcohol, and lauryl alcohol, but is not limited thereto.

In an exemplary embodiment, a weight ratio of the active ingredient to the cationic surfactant may be 1:50 to 50:1, preferably 1:10 to 40:1, more preferably 1:1 to 20:1, even more preferably 1:5 to 10:1, but is not limited thereto.

In an exemplary embodiment, the composition may be pH 3 to 6, preferably pH 3 to 5, more preferably pH 3.5 to 4.5, even more preferably pH 3.8 to 4.2, but is not limited thereto.

In an exemplary embodiment, the composition may not include a thickener. The thickener may be at least one selected from the group consisting of carbomers, acrylate-based polymers, xanthan gum, cellulose gum, tamarindus indica seed gum, PEG-150/decyl alcohol/SMDI copolymer, acrylates/steareth-20 methacrylate copolymer, acrylates/beheneth-25 methacrylate copolymer, and sodium acrylate/sodium acryloyldimethyl taurate copolymer, but is not limited thereto.

In an exemplary embodiment, the composition may not include PEG and/or PPG surfactants. For example, the PEG surfactant may be at least one selected from the group consisting of PEG-8 dimethicone, PEG-100 stearate, and PEG-20 glyceryl triisostearate, and the PPG surfactant may be PPG-8-ceteth-20 or PPG-13-decyltetradeceth-24, but is not limited thereto.

In an exemplary embodiment, the composition may be an emulsion or a cream formulation. The composition may be a formulation of an ampoule, a lotion, an essence, a cream, etc.

In an exemplary embodiment, a viscosity of the composition may be 2,000 cps or more.

In an exemplary embodiment, a strength of the composition may be in a range of 0.010 to 0.100 kgf or 0.010 to 0.080 kgf.

In an exemplary embodiment, the composition may be for skin pH adjustment.

In an exemplary embodiment, a skin stratum corneum applied with the composition may be pH 4 to 6.

In an exemplary embodiment, the pH may be maintained for 1 hour or more, preferably 6 hours or more, more preferably 12 hours or more, even more preferably 24 hours or more.

In an exemplary embodiment, the composition may not be removed after application. The composition may be a leave on formulation. The composition has low skin irritation, so it may be absorbed without rinsing off after application.

In an exemplary embodiment, the composition may be a cosmetic composition.

The present invention, in another perspective, relates to a method for adjusting skin pH, comprising applying an effective amount of an oil-in-water composition to a skin of a subject, based on the total oil-in-water composition weight, including an active ingredient of pH 4 or less in an amount of 0.1 wt % or more and less than 30 wt %; a surfactant in an amount of exceeding 2.6 wt %; a fatty acid and/or a fatty alcohol in an amount of exceeding 0.3 wt %; a chelating agent in an amount of less than 0.1 wt %; and a polyol, in which the surfactant includes a cationic amine-based surfactant in an amount of exceeding 0.6 wt % and a non-ionic surfactant in an amount of exceeding 0.1 wt %, in which the polyol is at least one polyol selected from the group consisting of sorbitol, maltitol, and xylitol, and when the cationic amine-based surfactant is a quaternary amine-based surfactant, the quaternary amine-based surfactant is behentrimonium methosulfate or distearyldimonium chloride.

The present invention, in yet another perspective, relates to a use of an active ingredient of pH 4 or less in an amount of 0.1 wt % or more and less than 30 wt %; a surfactant in an amount of exceeding 2.6 wt %; a fatty acid and/or a fatty alcohol in an amount of exceeding 0.3 wt %; a chelating agent in an amount of less than 0.1 wt %; and a polyol, for manufacturing an oil-in-water composition for skin pH adjustment, based on the total oil-in-water composition weight, in which the surfactant includes a cationic amine-based surfactant in an amount of exceeding 0.6 wt % and a non-ionic surfactant in an amount of exceeding 0.1 wt %, in which the polyol is at least one polyol selected from the group consisting of sorbitol, maltitol, and xylitol, and when the cationic amine-based surfactant is a quaternary amine-based surfactant, the quaternary amine-based surfactant is behentrimonium methosulfate or distearyldimonium chloride.

The present invention, in yet another perspective, relates to an active ingredient of pH 4 or less in an amount of 0.1 wt % or more and less than 30 wt %; a surfactant in an amount of exceeding 2.6 wt %; a fatty acid and/or a fatty alcohol in an amount of exceeding 0.3 wt %; a chelating agent in an amount of less than 0.1 wt %; and a polyol, for skin pH adjustment, based on a total weight, in which the surfactant includes a cationic amine-based surfactant in an amount of exceeding 0.6 wt % and a non-ionic surfactant in an amount of exceeding 0.1 wt %, in which the polyol is at least one polyol selected from the group consisting of sorbitol, maltitol, and xylitol, and when the cationic amine-based surfactant is a quaternary amine-based surfactant, the quaternary amine-based surfactant is behentrimonium methosulfate or distearyldimonium chloride.

The present invention, in yet another perspective, relates to a non-therapeutic use of an active ingredient of pH 4 or less in an amount of 0.1 wt % or more and less than 30 wt %; a surfactant in an amount of exceeding 2.6 wt %; a fatty acid and/or a fatty alcohol in an amount of exceeding 0.3 wt %; a chelating agent in an amount of less than 0.1 wt %; and a polyol, for skin pH adjustment, based on a total weight, in which the surfactant includes a cationic amine-based surfactant in an amount of exceeding 0.6 wt % and a non-ionic surfactant in an amount of exceeding 0.1 wt %, in which the polyol is at least one polyol selected from the group consisting of sorbitol, maltitol, and xylitol, and when the cationic amine-based surfactant is a quaternary amine-based surfactant, the quaternary amine-based surfactant is behentrimonium methosulfate or distearyldimonium chloride.

Hereinafter, the configuration and effects of the present invention will be described in more detail by way of examples. However, the following examples are provided only for exemplary purposes to help understanding of the present invention, and a scope and range of the present invention are not limited thereby.

### Examples

### Experimental Example 1. Manufacture of Examples and Comparative Examples

Examples 1 to 15 and Comparative Examples 1 to 12 were manufactured as follows (refer to Tables 1 to 3).

### Example 1

It was manufactured to include 8.5 wt % of fatty alcohol (2.5 wt % of glyceryl stearate, 6.0 wt % of cetearyl alcohol, 9.0 wt % of oil (7.0 wt % of ethylhexyl stearate, 2.0 wt % of dimethicone), 0.7 wt % of a tertiary amine-based surfactant (brassicamidopropyl dimethylamine), 2.0 wt % of a non-ionic surfactant (polyglyceryl-10 stearate), 7.0 wt % of a polyol (sorbitol), 0.01 wt % of a chelating agent (sodium metaphosphate), and 5.0 wt % of a low pH active ingredient (PHA).

### Example 2

It was manufactured identically to Example 1 except for including 20.0 wt % of the low pH active ingredient (PHA).

### Example 3

It was manufactured identically to Example 1 except for including 0.1 wt % of the low pH active ingredient (PHA).

### Example 4

It was manufactured identically to Example 1 except for including 2.0 wt % of the tertiary amine-based surfactant (brassicamidopropyl dimethylamine).

### Example 5

It was manufactured identically to Example 1 except for including 4.0 wt % of the tertiary amine-based surfactant (brassicamidopropyl dimethylamine).

### Example 6

It was manufactured identically to Example 1 except for not including a tertiary amine-based surfactant and including 2.0 wt % of a quaternary amine-based surfactant (behentrimonium methosulfate).

### Example 7

It was manufactured identically to Example 1 except for including 5.0 wt % of the non-ionic surfactant (polyglyceryl-10 stearate).

### Example 8

It was manufactured identically to Example 1 except for including 10.0 wt % of the non-ionic surfactant (polyglyceryl-10 stearate).

### Example 9

It was manufactured identically to Example 1 except for not including sodium metaphosphate as a chelating agent and including 0.01 wt % of disodium EDTA.

### Example 10

It was manufactured identically to Example 1 except for not including sodium metaphosphate as a chelating agent and including 0.05 wt % of disodium EDTA.

### Example 11

It was manufactured identically to Example 1 except for including 0.05 wt % of the chelating agent (sodium metaphosphate).

### Example 12

It was manufactured identically to Example 1 except for including 15.0 wt % of the polyol (sorbitol).

### Example 13

It was manufactured identically to Example 1 except for including 25.0 wt % of the polyol (sorbitol).

### Example 14

It was manufactured identically to Example 1 except for including 15.0 wt % of fatty alcohol (2.5 wt % of glyceryl stearate, 12.5 wt % of cetearyl alcohol).

### Example 15

It was manufactured identically to Example 1 except for including 15.0 wt % of fatty alcohol (9.0 wt % of glyceryl stearate, 6.0 wt % of cetearyl alcohol).

### Comparative Example 1

It was manufactured identically to Example 1 except for including 30.0 wt % of the low pH active ingredient (PHA).

### Comparative Example 2

It was manufactured identically to Example 1 except for including 0.6 wt % of the tertiary amine-based surfactant (brassicamidopropyl dimethylamine).

### Comparative Example 3

It was manufactured identically to Example 1 except for not including a tertiary amine-based surfactant and including 2.0 wt % of a quaternary amine-based surfactant (distearyldimonium chloride).

### Comparative Example 4

It was manufactured identically to Example 1 except for including 0.1 wt % of the non-ionic surfactant (polyglyceryl-10 stearate).

### Comparative Example 5

It was manufactured identically to Example 1 except for including 0.1 wt % of the chelating agent (sodium metaphosphate).

### Comparative Example 6

It was manufactured identically to Example 1 except for not including a chelating agent.

### Comparative Example 7

It was manufactured identically to Example 1 except for including 3.0 wt % of a polyol (glycerin) having a different number of hydroxyl groups (—OH) which are hydrophilic groups.

### Comparative Example 8

It was manufactured identically to Example 1 except for including 5.0 wt % of the polyol (glycerin).

### Comparative Example 9

It was manufactured identically to Example 1 except for including 7.0 wt % of the polyol (glycerin).

### Comparative Example 10

It was manufactured identically to Example 1 except for including [[5.0]]15.0 wt % of the polyol (glycerin).

### Comparative Example 11

It was manufactured identically to Example 1 except for including 0.3 wt % of fatty alcohol (0.1 wt % of glyceryl stearate, 0.2 wt % of cetearyl alcohol).

### Comparative Example 12

It was manufactured identically to Example 1 except for including 8.5 wt % of fatty alcohol (2.5 wt % of glyceryl stearate, 6.0 wt % of cetearyl alcohol) and not including a tertiary amine-based surfactant.

TABLE 1

| No. | CATEGORY1 | CATEGORY2 | CATEGORY3 | CATEGORY4 |
|---|---|---|---|---|
| 1 | FATTY ACID/ | FATTY | GLYCERYL STEARATE | |
| | FATTY | ALCOHOL | CETEARYL ALCOHOL | |
| | ALCOHOL | | BEHENYL ALCOHOL | |
| | | | ARACHIDYL ALCOHOL | |
| | | FATTY ACID | MYRISTIC ACID | |
| | | | STEARIC ACID | |
| | | | PALMITIC ACID | |
| | | | BEHENIC ACID | |
| 2 | OIL | HYDROCARBON- | DICAPRYLYL CARBONATE | |
| | | BASED | CAPRYLIC/CAPRIC TRIGLYCERIDE | |
| | | | squalane | |
| | | | ETHYLHEXYL STEARATE | |
| | | | HYDROGENATED POLYDECENE | |
| | | | DIETHOXYETHYL SUCCINATE | |
| | | SILICONE- | DIMETHICONE | |
| | | BASED | PHENYL TRIMETHICONE | |
| | | | DIPHENYLSILOXY PHENYL TRIMETHICONE | |
| | | | CAPRYLYL METHICONE | |
| 3 | SURFACTANT | CATIONIC | tertiary amine | Brassicamidopropyl Dimethylamine |
| | | AMINE-BASED | | RICINOLEAMIDO PROPYL |
| | | SURFACTANT | | DIMETHYLAMINE |
| | | | quaternary amine | BEHENTRIMONIUM METHOSULFATE |
| | | | | DISTEARYLDIMONIUM CHLORIDE |

| | |
|---|---|
| NON-IONIC | POLYGLYCERYL-10 STEARATE |
| SURFACTANT | POLYGLYCERYL-10 LAURATE |
| | POLYGLYCERYL-10 DIISOSTEARATE |
| | POLYGLYCERYL-10 MYRISTATE |
| | POLYGLYCERYL-10 OLEATE |
| | POLYGLYCERYL-3 METHYLGLUCOSE DISTEARATE |

| | | |
|---|---|---|
| 4 | POLYOL | glycerin |
| | | sorbitol |
| | | xylitol |
| | | maltitol |
| | | butylene glycol |
| | | dipropylene glycol |
| 5 | CHELATING | DISODIUM EDTA |
| | AGENT | SODIUM METAPHOSPHATE |
| | | SODIUM GLUCONATE |
| 6 | LOW pH | Gluconolactone (=PHA) |
| | ACTIVE | ASCORBIC ACID |
| | INGREDIENT | SALICYLIC ACID |
| | | GLYCOLIC ACID |
| | | LACTIC ACID |
| | | 3-O-Ethyl Ascorbic Acid |
| | | succinic acid |
| | | ferrulic acid |
| | | PHENYLBENZIMIDAZOLE SULFONIC ACID |
| | | Terephthalylidene Dicamphor Sulfonic Acid |

TABLE 2
Examples 1 to 15

| | | | | | | EXAM- | EXAM- | EXAM- | EXAM- | EXAM- | EXAM- | EXAM- |
|---|---|---|---|---|---|---|---|---|---|---|---|---|
| | CATE- | CATE- | CATE- | CATE- | EXAMPLE | PLE | PLE | PLE | PLE | PLE | PLE | PLE |
| | GORY1 | GORY2 | GORY3 | GORY4 | 1 | 2 | 3 | 4 | 5 | 6 | 7 | 8 |
| 1 | FATTY | FATTY | GLYCERYL | | 2.5 | 2.50 | 2.50 | 2.50 | 2.50 | 2.50 | 2.50 | 2.50 |
| | ACID/ | ALCOHOL | STEARATE | | | | | | | | | |
| | FATTY | | CETEARYL | | 6.00 | 6.00 | 6.00 | 6.00 | 6.00 | 6.00 | 6.00 | 6.00 |
| | ALCO- | | ALCOHOL | | | | | | | | | |
| | HOL | | | | | | | | | | | |
| 2 | OIL | HYDRO- | ETHYLHEXYL | | 7.00 | 7.00 | 7.00 | 7.00 | 7.00 | 7.00 | 7.00 | 7.00 |
| | | CARBON- | STEARATE | | | | | | | | | |
| | | BASED | | | | | | | | | | |
| | | SILICONE- | DIMETHICONE | | 2.00 | 2.00 | 2.00 | 2.00 | 2.00 | 2.00 | 2.00 | 2.00 |
| | | BASED | | | | | | | | | | |
| 3 | SURFAC- | CATIONIC | tertiary | Brassic- | 0.70 | 0.70 | 0.70 | 2.00 | 4.00 | | 0.70 | 0.70 |
| | TANT | AMINE- | amine | amido- | | | | | | | | |
| | | BASED | | propyl | | | | | | | | |
| | | SURFAC- | | Dimethyl- | | | | | | | | |
| | | TANT | | amine | | | | | | | | |
| | | | quater- | BEHENTRI- | | | | | | 2.00 | | |
| | | | nary | MONIUM | | | | | | | | |
| | | | amine | METHO- | | | | | | | | |
| | | | | SULFATE | | | | | | | | |
| | | NON- | POLYGLY- | | 2.00 | 2.00 | 2.00 | 2.00 | 2.00 | 2.00 | 5.00 | 10.00 |
| | | IONIC | CERYL-10 | | | | | | | | | |
| | | SURFAC- | STEARATE | | | | | | | | | |
| | | TANT | | | | | | | | | | |
| 4 | POLYOL | sorbitol | | | 7.00 | 7.00 | 7.00 | 7.00 | 7.00 | 7.00 | 7.00 | 7.00 |
| 5 | CHEL- | DISODIUM EDTA | | | | | | | | | | |
| | ATING | SODIUM | | | 0.01 | 0.01 | 0.01 | 0.01 | 0.01 | 0.01 | 0.01 | 0.01 |
| | AGENT | METAPHOSPHATE | | | | | | | | | | |
| 6 | LOW pH | Gluconolactone (=PHA) | | | 5.00 | 20.00 | 5.00 | 5.00 | 0.10 | 5.00 | 5.00 | 5.00 |
| | ACTIVE | | | | | | | | | | | |
| | INGRE- | | | | | | | | | | | |
| | DIENT | | | | | | | | | | | |

| 7 | OTHERS | Water | | TO-100 | | | | | | | |
|---|---|---|---|---|---|---|---|---|---|---|---|
| | | | | | EXAM- | EXAM- | EXAM- | EXAM- | EXAM- | EXAM- | EXAM- |
| | CATE- | | | CATE- | PLE | PLE | PLE | PLE | PLE | PLE | PLE |
| | GORY1 | CATEGORY2 | CATEGORY3 | GORY4 | 9 | 10 | 11 | 12 | 13 | 14 | 15 |
| 1 | FATTY | FATTY | GLYCERYL | | 2.50 | 2.50 | 2.50 | 2.50 | 2.50 | 2.50 | 9.00 |
| | ACID/ | ALCOHOL | STEARATE | | | | | | | | |
| | FATTY | | CETEARYL | | 6.00 | 6.00 | 6.00 | 6.00 | 6.00 | 12.50 | 6.00 |
| | ALCOHOL | | ALCOHOL | | | | | | | | |
| 2 | OIL | HYDRO- | ETHYLHEXYL | | 7.00 | 7.00 | 7.00 | 7.00 | 7.00 | 7.00 | 7.00 |
| | | CARBON- | STEARATE | | | | | | | | |
| | | BASED | | | | | | | | | |
| | | SILICONE- | DIMETHICONE | | 2.00 | 2.00 | 2.00 | 2.00 | 2.00 | 2.00 | 2.00 |
| | | BASED | | | | | | | | | |
| 3 | SURFAC- | CATIONIC | tertiary | Brassic- | 0.70 | 0.70 | 0.70 | 0.70 | 0.70 | 0.70 | 0.70 |
| | TANT | AMINE- | amine | amido- | | | | | | | |
| | | BASED | | propyl | | | | | | | |
| | | SURFAC- | | Dimethyl- | | | | | | | |
| | | TANT | | amine | | | | | | | |
| | | | quaternary | BEHENTRI- | | | | | | | |
| | | | amine | MONIUM | | | | | | | |
| | | | | METHO- | | | | | | | |
| | | | | SULFATE | | | | | | | |
| | | NON-IONIC | POLYGLY-CERYL-10 | | 2.00 | 2.00 | 2.00 | 2.00 | 2.00 | 2.00 | 2.00 |
| | | SURFAC- | STEARATE | | | | | | | | |
| | | TANT | | | | | | | | | |
| 4 | POLYOL | sorbitol | | | 7.00 | 7.00 | 7.00 | 15.00 | 25.00 | 7.00 | 7.00 |
| 5 | CHELAT- | DISODIUM EDTA | | | 0.01 | 0.05 | | | | | |
| | ING | SODIUM | | | | | 0.05 | 0.01 | 0.01 | 0.01 | 0.01 |
| | AGENT | METAPHOSPHATE | | | | | | | | | |
| 6 | LOW pH | Gluconolactone (=PHA) | | | 5.00 | 5.00 | 5.00 | 5.00 | 5.00 | 5.00 | 5.00 |
| | ACTIVE | | | | | | | | | | |
| | INGRE- | | | | | | | | | | |
| | DIENT | | | | | | | | | | |

| | | | |
|---|---|---|---|
| 7 | OTHERS | Water | TO-100 |

TABLE 3
Comparative Examples 1 to 12

| | | | | | COM- | COM- | COM- | COM- | COM- | COM- |
|---|---|---|---|---|---|---|---|---|---|---|
| | | | | | PARA- | PARA- | PARA- | PARA- | PARA- | PARA- |
| | | | | | TIVE | TIVE | TIVE | TIVE | TIVE | TIVE |
| | | | | | EXA- | EXA- | EXA- | EXA- | EXA- | EXA- |
| | CATE- | CATE- | CATE- | CATE- | MPLE | MPLE | MPLE | MPLE | MPLE | MPLE |
| | GORY1 | GORY2 | GORY3 | GORY4 | 1 | 2 | 3 | 4 | 5 | 6 |
| 1 | FATTY | FATTY | GLYCERYL | | 2.50 | 2.50 | 2.50 | 2.50 | 2.50 | 2.50 |
| | ACID/ | ALCOHOL | STEARATE | | | | | | | |
| | FATTY | | CETEARYL | | 6.00 | 6.00 | 6.00 | 6.00 | 6.00 | 6.00 |
| | ALCOHOL | | ALCOHOL | | | | | | | |
| 2 | OIL | HYDRO- | ETHYLHEXYL | | 7.00 | 7.00 | 7.00 | 7.00 | 7.00 | 7.00 |
| | | CARBON- | STEARATE | | | | | | | |
| | | BASED | | | | | | | | |
| | | SILICONE- | DIMETHICONE | | 2.00 | 2.00 | 2.00 | 2.00 | 2.00 | 2.00 |
| | | BASED | | | | | | | | |
| 3 | SURFAC- | CATIONIC | tertiary | Brassica- | 0.70 | 0.60 | — | 0.70 | 0.70 | 0.70 |
| | TANT | AMINE- | amine | midopropyl | | | | | | |
| | | BASED | | Dimethyl- | | | | | | |
| | | SURFAC- | | amine | | | | | | |
| | | TANT | quater- | DISTEARYL- | | | 2.00 | | | |
| | | | nary | DIMONIUM | | | | | | |
| | | | amine | CHLORIDE | | | | | | |
| | | NON- | POLYGLYCERYL-10 | | 2.00 | 2.00 | 2.00 | 0.10 | 2.00 | 2.00 |
| | | IONIC | STEARATE | | | | | | | |
| | | SURFAC | | | | | | | | |
| | | TANT | | | | | | | | |
| 4 | POLYOL | glycerin | | | | | | | | |
| | | sorbitol | | | 7.00 | 7.00 | 7.00 | 7.00 | 7.00 | |
| 5 | CHELAT- | SODIUM METAPHOSPHATE | | | 0.01 | 0.01 | 0.01 | 0.01 | 0.10 | 0.01 |
| | ING | | | | | | | | | |
| | AGENT | | | | | | | | | |
| 6 | LOW pH | Gluconolactone (=PHA) | | | 30.00 | 5.00 | 5.00 | 5.00 | 5.00 | 5.00 |
| | ACTIVE | | | | | | | | | |
| | INGRE- | | | | | | | | | |
| | DIENT | | | | | | | | | |
| 7 | OTHERS | Water | | | TO-100 | | | | | |
| | | | | | COM- | COM- | COM- | COM- | COM- | COM- |
| | | | | | PARA- | PARA- | PARA- | PARA- | PARA- | PARA- |
| | | | | | TIVE | TIVE | TIVE | TIVE | TIVE | TIVE |
| | | | | | EXA- | EXA- | EXA- | EXA- | EXA- | EXA- |
| | CATE- | CATE- | CATE- | CATE- | MPLE | MPLE | MPLE | MPLE | MPLE | MPLE |
| | GORY1 | GORY2 | GORY3 | GORY4 | 7 | 8 | 9 | E10 | 11 | 12 |
| 1 | FATTY | FATTY | GLYCERYL | | 2.50 | 2.50 | 2.50 | 2.50 | 0.10 | 2.50 |
| | ACID/ | ALCOHOL | STEARATE | | | | | | | |
| | FATTY | | CETEARYL | | 6.00 | 6.00 | 6.00 | 6.00 | 0.20 | 6.00 |
| | ALCOHOL | | ALCOHOL | | | | | | | |
| 2 | OIL | HYDRO- | ETHYLHEXYL | | 7.00 | 7.00 | 7.00 | 7.00 | 7.00 | 7.00 |
| | | CARBON- | STEARATE | | | | | | | |
| | | BASED | | | | | | | | |
| | | SILICONE- | DIMETHICONE | | 2.00 | 2.00 | 2.00 | 2.00 | 2.00 | 2.00 |
| | | BASED | | | | | | | | |
| 3 | SURFAC- | CATIONIC | tertiary | Brassica- | 0.70 | 0.70 | 0.70 | 0.70 | 0.70 | — |
| | TANT | AMINE- | amine | midopropyl | | | | | | |
| | | BASED | | Dimethyl- | | | | | | |
| | | SURFAC- | | amine | | | | | | |
| | | TANT | quater- | DISTEARYL- | | | | | | |
| | | | nary | DIMONIUM | | | | | | |
| | | | amine | CHLORIDE | | | | | | |
| | | NON- | POLYGLYCERYL-10 | | 2.00 | 2.00 | 2.00 | 2.00 | 2.00 | 2.00 |
| | | IONIC | STEARATE | | | | | | | |
| | | SURFAC | | | | | | | | |
| | | TANT | | | | | | | | |
| 4 | POLYOL | glycerin | | | 3.00 | 5.00 | 7.00 | 15.00 | | |
| | | sorbitol | | | | | | | 7.00 | 7.00 |
| 5 | CHELAT- | SODIUM METAPHOSPHATE | | | 0.01 | 0.01 | 0.01 | 0.01 | 0.01 | 0.01 |
| | ING | | | | | | | | | |
| | AGENT | | | | | | | | | |
| 6 | LOW pH | Gluconolactone (=PHA) | | | 5.00 | 5.00 | 5.00 | 5.00 | 5.00 | 5.00 |
| | ACTIVE | | | | | | | | | |
| | INGRE- | | | | | | | | | |
| | DIENT | | | | | | | | | |

| | | | |
|---|---|---|---|
| 7 | OTHERS | Water | TO-100 |

TABLE 4

| CHARAC- | EXAMPLE | EXAMPLE | EXAMPLE | EXAMPLE | EXAMPLE | EXAMPLE | EXAMPLE | EXAMPLE |
|---|---|---|---|---|---|---|---|---|
| TERISTIC | 1 | 2 | 3 | 4 | 5 | 6 | 7 | 8 |
| HARDNESS | 0.055 ± 0.010 kgf | | | 0.060 ± 0.010 kgf | | | 0.055 ± 0.010 kgf | |
| [kgf] | | | | | | | | |
| FORMULA- | 3~5 | | | 3~5 | | | 3~5 | |
| TION pH | | | | | | | | |
| MANUFAC- | GOOD | GOOD | GOOD | GOOD | GOOD | GOOD | GOOD | GOOD |
| TURING | | | | | | | | |
| RESULT | | | | | | | | |
| | | EXAMPLE | EXAMPLE | EXAMPLE | EXAMPLE | EXAMPLE | EXAMPLE | EXAMPLE |

| CHARACTERISTIC | 9 | 10 | 11 | 12 | 13 | 14 | 15 |
|---|---|---|---|---|---|---|---|
| HARDNESS [kgf] | 0.055 ± 0.010 kgf | | | 0.055 ± 0.010 kgf | | 0.085 ± 0.010 kgf | |
| FORMULATION pH | 3~5 | | | 3~5 | | 3~5 | |
| MANUFACTURING | GOOD | GOOD | GOOD | GOOD | GOOD | GOOD | GOOD |
| RESULT | | | | | | | |

TABLE 5

| CHARAC- | COMPARATIVE | COMPARATIVE | COMPARATIVE | COMPARATIVE | COMPARATIVE | COMPARATIVE |
|---|---|---|---|---|---|---|
| TERISTIC | EXAMPLE 1 | EXAMPLE 2 | EXAMPLE 3 | EXAMPLE 4 | EXAMPLE 5 | EXAMPLE 6 |
| MANUFAC- | SEPARATION | SEPARATION | SEPARATION | SEPARATION | SEPARATION | SEPARATION |
| TURING | OR POOR | OR POOR | OR POOR | OR POOR | OR POOR | OR POOR |
| RESULT | STABILITY | STABILITY | STABILITY | STABILITY | STABILITY | STABILITY |
| CHARAC- | COMPARATIVE | COMPARATIVE | COMPARATIVE | COMPARATIVE | COMPARATIVE | COMPARATIVE |
| TERISTIC | EXAMPLE 7 | EXAMPLE 8 | EXAMPLE 9 | EXAMPLE 10 | EXAMPLE 11 | EXAMPLE 12 |
| MANUFAC- | SEPARATION | SEPARATION | SEPARATION | SEPARATION | SEPARATION | SEPARATION |
| TURING | OR POOR | OR POOR | OR POOR | OR POOR | OR POOR | OR POOR |
| RESULT | STABILITY | STABILITY | STABILITY | STABILITY | STABILITY | STABILITY |

### Experimental Example 2. Comparison of Formulation Stability and Feeling of Use According to Low pH Active Ingredient Content

As a result of comparing formulation stability and feeling of use according to a low pH active ingredient content, it was confirmed that Example 1 (5.0 wt % of PHA), Example 2 (20.0 wt % of PHA), and Example 3 (0.1 wt % of PHA) all are excellent in formulation stability and feeling of use.

On the other hand, Comparative Example 1 (30.0 wt % of PHA) showed poor stability/a separated form and a negative feeling of use (for example, stickiness). More specifically, it was confirmed that, when the low pH active ingredient is included in an ultra-high content, a basic neutralizing agent was used in a high content, and thus negative attributes are induced in formulation stability and feeling of use.

Accordingly, thereafter, the composition was manufactured including the low pH active ingredient content in 0.1% or more to less than 30 wt %.

### Experimental Example 3. Comparison of Formulation Stability and Feeling of Use According to Surfactant Type and Content

As a result of comparing formulation stability and feeling of use according to a surfactant type and content, it was confirmed that Example 4 (2.0 wt % of a tertiary amine-based surfactant) is excellent in formulation stability and feeling of use. In Example 5 (4.0 wt % of a tertiary amine-based surfactant) and Example 6 (2.0 wt % of a quaternary amine-based surfactant behentrimonium methosulfate), a formulation stability risk was increased, and some negative initial feeling of use (for example, excessively oily immediately after use) appeared, but it was confirmed that formulation stability and feeling of use are generally good.

On the other hand, Comparative Example 2 (0.6 wt % of a tertiary amine-based surfactant) and Comparative Example 3 (2.0 wt % of a quaternary amine-based surfactant distearyldimonium chloride) showed poor stability/a separated form.

Accordingly, thereafter, the composition was manufactured including a cationic amine-based surfactant content in exceeding 0.6 wt %. In addition, when the cationic amine-based surfactant is a quaternary amine-based surfactant, behentrimonium methosulfate was used.

In addition, in Example 7 (5.0 wt % of a non-ionic surfactant), some negative initial feeling of use (for example, not being fresh immediately after use) and a white cast phenomenon appeared, but it was confirmed that formulation stability and feeling of use are generally good. In Example 8 (10.0 wt % of a non-ionic surfactant), some negative initial feeling of use (for example, stickiness immediately after use) and a white cast phenomenon appeared, but it was confirmed that formulation stability and feeling of use are generally good.

On the other hand, Comparative Example 4 (0.1 wt % of a non-ionic surfactant) showed poor stability/a separated form.

Accordingly, the composition was manufactured including a non-ionic surfactant content in exceeding 0.1 wt %.

### Experimental Example 4. Comparison of Formulation Stability According to Chelating Agent Type and Content

As a result of comparing formulation stability and feeling of use according to a chelating agent type and content, it was confirmed that Example 9 (0.01 wt % of disodium EDTA), Example 10 (0.05 wt % of disodium EDTA), and Example 11 (0.05 wt % of sodium metaphosphate) are generally good in formulation stability and feeling of use.

On the other hand, Comparative Example 5 (0.1 wt % of sodium metaphosphate) showed poor stability/a separated form.

Accordingly, thereafter, the composition was manufactured including a chelating agent content in less than 0.1 wt %.

### Experimental Example 5. Comparison of Formulation Stability According to Polyol Type and Content

It was confirmed that Example 12 (15 wt % of sorbitol) and Example 13 (25 wt % of sorbitol) are excellent in formulation stability and feeling of use, and a high moisturizing effect appeared.

On the other hand, in Comparative Example 6 (not including a polyol), it was confirmed that a state is poor as aggregates are formed immediately after manufacturing, and Comparative Example 7 (3 wt % of glycerin), Comparative Example 8 (5 wt % of glycerin), Comparative Example 9 (7 wt % of glycerin), and Comparative Example 10 (15 wt % of glycerin) showed poor stability/a separated form.

Accordingly, thereafter, the composition was manufactured including sorbitol.

### Experimental Example 6. Comparison of Formulation Stability According to Fatty Acid or Fatty Alcohol Type and Content

As a result of comparing formulation stability and feeling of use according to a low pH active ingredient content, Example 14 and Example 15 (15 wt % of fatty alcohol) showed a stiff and matte feeling of use as they include a wax-type fatty alcohol (or a fatty acid may be used) in a high concentration, but formulation stability was excellent.

On the other hand, Comparative Example 11 (0.3 wt % of fatty alcohol) showed poor stability/a separated form.

Accordingly, thereafter, the composition was manufactured including a fatty acid and/or a fatty alcohol in exceeding 0.3 wt %.

### Experimental Example 7. Viscosity or Strength Analysis

For strength analysis, hardness (kgf) was measured using Sun Rheometer (Compac-100) equipment. A measurement method was performed in a manner of compressing a sample to a depth of 2 cm at a speed of 10 cm/min using a cylinder of 2 cm diameter with 2 kg weight, and measuring kilogram-force (kgf) received by the sample during this process.

Viscosity measurement was carried out using a Brookfield viscometer on a day of manufacturing or a next day under a condition of a content temperature of 30° C. Measurement was performed for 2 minutes at 12 rpm using a No. 63 or No. 64 spindle.

As a result, an Experimental Example group showed a high viscosity and high strength formulation and a non-sticky and fresh texture compared to a Comparative Example group. Specifically, a strength (hardness) range appeared as 0.010 to 0.100 kgf, and viscosity showed a relatively high value, and physical properties maintained stably could be confirmed.

### Experimental Example 8. Evaluation of Low pH Persistence

To evaluate low pH persistence, after applying a cosmetic (0.1 g) to a skin (forearm, 2 cm×2 cm area), pH before application and after application was measured using a pH meter.

As a result, as shown in FIG. 2, in both the cosmetic composition including 3 wt % of PHA and the cosmetic composition including 10 wt % of PHA, pH effectively decreased to pH 4.5 or less immediately after use, and thereafter, it was confirmed that a low pH is maintained for a long period (24 hours).

### Experimental Example 9. Sensory Evaluation

For sensory evaluation, while applying a cosmetic (0.1 g) to a skin (forearm, 2 cm×2 cm area), feeling of use was evaluated by scoring, and further, after leaving for 3 minutes after application, a score for feeling of use was additionally evaluated.

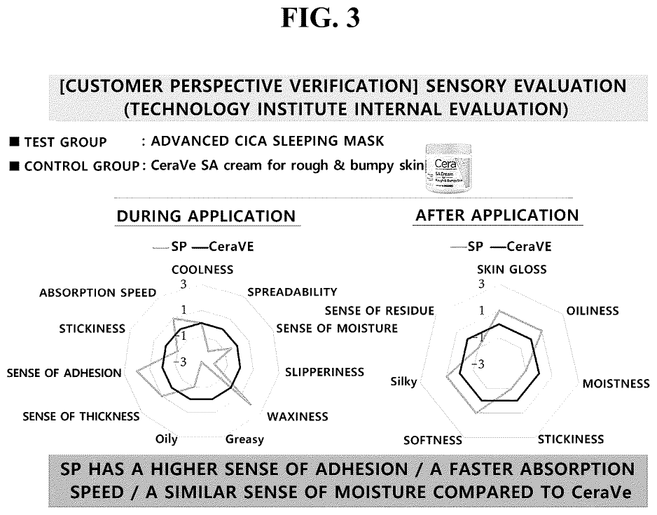

As a result, as shown in FIG. 3, it was confirmed that the Experimental Example group shows a higher sense of adhesion and a faster absorption speed compared to the Comparative Example group.

### Experimental Example 10. Efficacy Analysis of Low pH Active Ingredient

Efficacy of a low pH active ingredient was evaluated for a total of 4 research subjects (n=4, women in their 30s). Image capturing was performed using VISIA equipment on a forearm part, and measurement was made at time points of immediately after henna dyeing, a 3rd day of product use, and a 6th day. Samples used in the experiment were composed of a cosmetic composition (test group) including 3% of PHA (gluconolactone) and a cosmetic composition (control group) including 1% of AHA (glycolic acid).

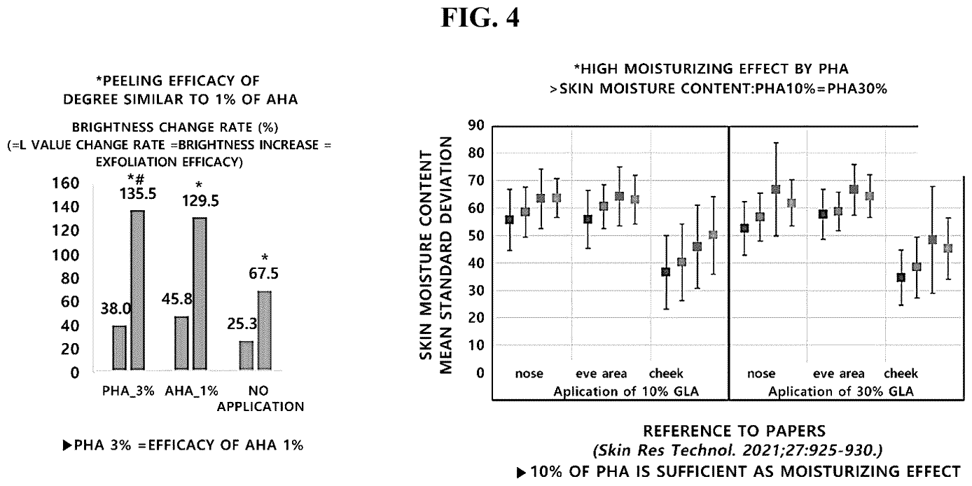

As a result, as shown in FIG. 4, it was confirmed that the cosmetic composition including 3% of PHA showed excellent peeling (exfoliation) efficacy, and the cosmetic composition including 10% or more of PHA showed an excellent moisturizing effect.

What is claimed is:

1. An oil-in-water composition, comprising:
an active ingredient of pH 4 or less in an amount of 0.1 wt % or more and less than 30 wt %;
a surfactant in an amount of exceeding 2.6 wt %;
a fatty acid and/or a fatty alcohol in an amount of exceeding 0.3 wt %;
a chelating agent in an amount of less than 0.1 wt %; and
a polyol, based on a total oil-in-water composition weight,
wherein the surfactant comprises a cationic amine-based surfactant in an amount of exceeding 0.6 wt % and a non-ionic surfactant in an amount of exceeding 0.1 wt %,
wherein the polyol is at least one polyol selected from the group consisting of sorbitol, maltitol, and xylitol, and when the cationic amine-based surfactant is a quaternary amine-based surfactant, the quaternary amine-based surfactant is behentrimonium methosulfate or distearyldimonium chloride.

2. The composition of claim 1, wherein the active ingredient of pH 4 or less is at least one selected from the group consisting of poly-hydroxy-acid (PHA), beta-hydroxy-acid (BHA), alpha-hydroxy-acid (AHA), pure vitamin C (ascorbic acid), vitamin C derivatives (ethyl ascorbic acid), succinic acid, ferulic acid, aspartic acid, glutamic acid, phenylbenzimidazole sulfonic acid, and terephthalylidene dicamphor sulfonic acid.

3. The composition of claim 1, wherein the cationic surfactant is a tertiary amine-based surfactant or a quaternary amine-based surfactant.

4. The composition of claim 1, wherein a weight ratio of the active ingredient to the cationic surfactant is 1:50 to 50:1.

5. The composition of claim 1, wherein the composition is pH 3 to 6.

6. The composition of claim 1, wherein the composition does not comprise a thickener.

7. The composition of claim 1, wherein the composition does not comprise PEG and/or PPG surfactants.

8. The composition of claim 1, wherein the composition is an emulsion or a cream formulation.

9. The composition of claim 1, wherein a viscosity of the composition is 2,000 cps or more.

10. The composition of claim 1, wherein a strength of the composition is 0.010 to 0.100 kgf.

11. A method for adjusting skin pH, comprising applying an effective amount of the oil-in-water composition of claim 1 to a skin of a subject.

12. The method of claim 11, wherein a skin stratum corneum applied with the composition is pH 4 to 6.

13. The method of claim 12, wherein the pH is maintained for 1 hour or more.

14. The method of claim 11, wherein the composition is not removed after application.

15. The composition of claim 1, wherein the composition is a cosmetic composition.
